(19) United States
(12) Patent Application Publication
(10) Pub. No.: US 2024/0024028 A1
(43) Pub. Date: Jan. 25, 2024

(54) SYSTEMS AND METHODS FOR VERIFYING A POSE OF A TARGET

(71) Applicant: Mazor Robotics Ltd., Caesarea (IL)

(72) Inventors: Gal Barazani, Tirat Carmel (IL); Nir Ofer, Tel Aviv-Yafo (IL); Itay Jerby, Netanya (IL); Ofir Dahan, Haifa (IL); Mark Raikhman, Haifa (IL)

(21) Appl. No.: 17/870,242

(22) Filed: Jul. 21, 2022

(51) Int. Cl. A61B 34/10; A61B 34/20; A61B 34/00; B25J 9/16

(52) U.S. Cl. CPC A61B 34/10; A61B 34/20; A61B 34/70; B25J 9/1697; B25J 9/1692; B25J 9/1682; A61B 2034/107; A61B 2034/2065

(57) ABSTRACT

Systems and methods for verifying a pose of a target may include causing a robotic arm to contact a target with a verification tool to yield first pose information of the target and receiving second pose information of the target. The pose of the target may be verified based on the first pose information and the second pose information.

### BACKGROUND

The present disclosure is generally directed to verifying a pose of a target, and relates more particularly to verifying a pose of a target using a verification tool.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A system for verifying a pose of a target during a surgical operation according to at least one embodiment of the present disclosure comprises a robotic arm; a verification tool coupled to the robotic arm; a processor; and a memory storing data for processing by the processor, the data, when processed, causes the processor to: cause the robotic arm to contact a target with the verification tool to yield first pose information of the target; receive second pose information of the target; and verify the pose of the target based on the first pose information and the second pose information.

Any of the aspects herein, wherein the second pose information of the target is received from another robotic arm or a registration process.

Any of the aspects herein, wherein the memory stores further data for processing by the processor that, when processed, causes the processor to: perform the registration process; receive the second pose information from the registration process; determine a difference between the first pose information and the second pose information of the target; and adjust the registration by shifting the second pose information of the target based on the difference.

Any of the aspects herein, wherein the memory stores further data for processing by the processor that, when processed, causes the processor to: perform the registration process; receive the second pose information from the registration process; and verify the registration based on the first pose information and the second pose information.

Any of the aspects herein, wherein the target comprises another robotic arm or a target anatomical element.

Any of the aspects herein, wherein verifying the pose of the target verifies a calibration of the robotic arm.

Any of the aspects herein, wherein the verification tool comprises a probe.

Any of the aspects herein, wherein the probe is configured to sense when the robotic arm contacts the target, and wherein the memory stores further data for processing by the processor that, when processed, causes the processor to: cause the robotic arm to move towards the target; receive a signal from the probe corresponding to the probe contacting the target; cause the robotic arm to pause movement; receive pose information of the robotic arm; and determine the first pose information of the target based on the pose information of the robotic arm.

Any of the aspects herein, wherein the probe comprises a force sensor and wherein the probe sends a signal corresponding to the probe contacting the target when the force sensor measures a force that meets a predetermined force threshold.

Any of the aspects herein, wherein determining the first pose information comprises combining the pose of the robotic arm with a known length of the probe.

Any of the aspects herein, wherein the memory stores further data for processing by the processor that, when processed, causes the processor to receive third pose information; and verify the pose of the target based on the first pose information, the second pose information, and the third pose information.

Any of the aspects herein, wherein verifying the pose of the target occurs prior to a surgical step.

A system for verifying a pose of a target during a surgical operation according to at least one embodiment of the present disclosure comprises a verification tool; a processor; and a memory storing data for processing by the processor, the data, when processed, causes the processor to: receive first pose information of the target using the verification tool; receive second pose information of the target; and verifying the pose of the target based on the first pose information and the second pose information.

Any of the aspects herein, wherein the target comprises a robotic arm or a target anatomical element.

Any of the aspects herein, wherein the verification tool comprises a probe.

Any of the aspects herein, wherein determining the first pose information comprises combining the pose of the robotic arm with a known length of the probe.

Any of the aspects herein, wherein the memory stores further data for processing by the processor that, when processed, causes the processor to receive third pose information; and verify the pose of the target based on the first pose information, the second pose information, and the third pose information.

Any of the aspects herein, wherein the memory stores further data for processing by the processor that, when processed, causes the processor to: perform a registration process; receive the second pose information from the registration process; determine a difference between the first pose information and the second pose information of the target; and adjust the registration by shifting the second pose information of the target based on the difference.

Any of the aspects herein, wherein the memory stores further data for processing by the processor that, when processed, causes the processor to: perform a registration process; receive the second pose information from the registration process; and verify the registration based on the first pose information and the second pose information.

A system for verifying a pose of a target according to at least one embodiment of the present disclosure comprises a processor; and a memory storing data for processing by the processor, the data, when processed, causes the processor to: cause a robotic arm to contact a target with a verification tool to yield first pose information of the target; cause the robotic arm to contact a target with the verification tool to yield second pose information of the target; cause the robotic arm to contact a target with the verification tool to yield third pose information of the target; and verifying the pose of the target based on the first pose information, the second pose information, and the third pose information.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram of a robotic system according to at least one embodiment of the present disclosure;

FIG. 3 is a flowchart according to at least one embodiment of the present disclosure;

FIG. 4 is a flowchart according to at least one embodiment of the present disclosure; and

FIG. 5 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

When performing a surgical operation using a robotic system (whether to perform a robotically assisted operation or an autonomous operation), it is desirable to know or track a location of an operating bed or table and/or patient anatomy being operated on relative to the robotic system as the patient anatomy may move relative to the bed and/or the robotic system. For example, the operating bed or the patient may be pushed and may result in a deflection between the patient and the robotic system. Additionally, after a registration process is completed, it is difficult to determine if a shift has occurred between coordinate systems (e.g., a patient coordinate system, a robotic coordinate system, etc.). Further, prior to leaving a manufacturing facility, two or more robotic arms of the robotic system may be calibrated relative to each other. This calibration may become corrupted during shipment and/or installation of the robotic system.

At least one embodiment according to the present disclosure may include a robotic system wherein one or more verification tools (e.g., probes) are attached to an end unit of one or more robotic arms of the robotic system. The verification tool and a first robotic arm can be used to perform a verification process before or during a surgical operation. The verification process performed by the verification tool may include evaluating a location or pose of a second robotic arm. With one or more measurements from the verification tool and the first robotic arm, the robotic calibration can be verified using the one or more measurements. Further, the verification process can be used to measure a location or pose of a segment point of a patient's anatomy and compare the segment point to the registration. With a few segment points, a deviation of the patient anatomy to the registration can be determined and can be compensated for without re-registration. This is advantageous as the registration process is cumbersome and time consuming. Thus, by minimizing the need to perform re-registrations, an overall operating time may be decreased. The verification process also can be used as a safety layer to validate an accuracy of the robotic system and/or the registration before and during a surgical operation. It will be appreciated that such verification processes can be performed multiple times during a surgical operation (e.g., prior to, during, or after a surgical step).

Embodiments of the present disclosure provide technical solutions to one or more of the problems of (1) verifying a registration process, (2) verifying a robot calibration, (3) verifying a pose of a target anatomical element, and (4) increasing patient and operating team safety.

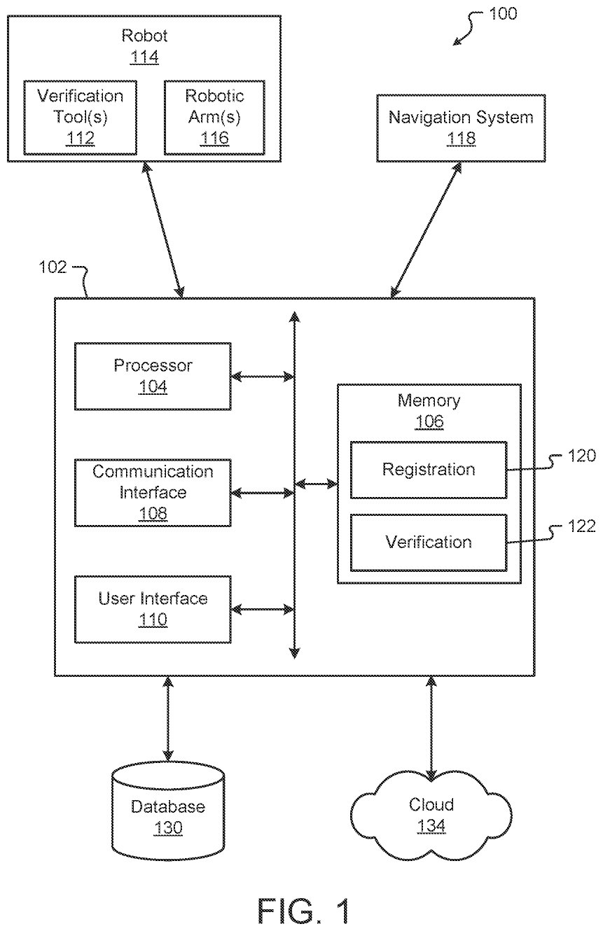

Turning first to FIG. 1, a block diagram of a system 100 according to at least one embodiment of the present disclosure is shown. The system 100 may be used to verify a pose of a target and/or carry out one or more other aspects of one or more of the methods disclosed herein. The system 100 comprises a computing device 102, a robot 114, a navigation system 118, a database 130, and/or a cloud or other network 134. Systems according to other embodiments of the present disclosure may comprise more or fewer components than the system 100. For example, the system 100 may not include the robot 114, the navigation system 118, one or more components of the computing device 102, the database 130, and/or the cloud 134.

The computing device 102 is shown to include a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the robot 114 (or a verification tool 112 of the robot 114), the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300, 400, and/or 500 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable registration 120 and/or verification 122. Such content, if provided as in instruction, may, in some embodiments, be organized into one or more applications, modules, packages, layers, or engines.

The registration 120, when executed by the processor 104, enables the processor 104 (or a processor of the navigation system 118) to perform a registration process between a robotic coordinate space and a patient coordinate space. In other embodiments, the registration process may be performed between a navigation coordinate space and a patient coordinate space, between a navigation coordinate space and a robotic coordinate space, and/or between an image coordinate space and one or more of a robotic coordinate space, a patient coordinate space, and/or a navigation coordinate space. The registration 120 may enable the processor 104 to map, transform, or otherwise correlate a particular location with respect to one coordinate space to the particular location with respect to another coordinate space.

The verification 122, when executed by the processor 104, enables the processor 104 (or a processor of the navigation system 118) to process pose information (received from, for example, the robot 114) of a target (e.g., a robotic arm, a target anatomical element, an operating table, etc.) for the purpose of, for example, verifying a pose of the target. The verification 122 may verify the pose of the target by comparing first pose information of the target received from the robot 114 and second pose information of the target received from, for example, a registration process and/or the robot 114. More specifically, in some embodiments, the verification 122 may determine a pose difference between the first pose information and the second pose information. The verification 122 may then validate the pose of the target in response to determining that the pose difference is less than a pose threshold.

Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the robot 114, the database 130, and/or the cloud 134.

The computing device 102 may also comprise a communication interface 108. The communication interface 108 may be used for receiving image data or other information from an external source (such as the robot 114, the navigation system 118, the database 130, the cloud 134, and/or any other system or component not part of the system 100), and/or for transmitting instructions, images, or other information to an external system or device (e.g., another computing device 102, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The robot 114 may be any surgical robot or surgical robotic system. The robot 114 may be or comprise, for example, the Mazor X™ Stealth Edition robotic guidance system. The robot 114 may be configured to position a tool (such as, for example, a verification tool 112) at one or more precise position(s) and orientation(s), and/or to return the tool to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver a tool. Each robotic arm 116 may be positionable independently of the other robotic arm. The robotic arms 116 may be controlled in a single, shared coordinate space, or in separate coordinate spaces. Further, the robotic arms 116 may be calibrated relative to each other.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, a surgical tool or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm). The sensor(s) may be a position sensor, a proximity sensor, a magnetometer, an accelerometer, a linear encoder, a rotary encoder, or an incremental encoder.

The robot 114 may comprise one or more verification tools 112. The verification tool(s) 112 may be disposed at an end of the robotic arm 116. In other instances, the verification tool(s) 112 may be positioned on any part of the robotic arm 116 or the robot 114. In still other instances, more than one verification tool 112 may be attached or coupled to the robotic arm 116. In some embodiments, the verification tool(s) 112 may be sterilized so as enable contact of a patient anatomy during a surgical operation.

The verification tool 112 may be configured to detect when the robotic arm 116 is contacting a target (e.g., another robotic arm, a target anatomical element, an operating bed, or any other component). The verification tool 112 may comprise a force sensor or contact sensor configured to detect a force applied on the robotic arm 116 (e.g., a probe). In such embodiments, the force sensor may be used to determine when the robotic arm 116 is contacting the target based on a detection of the force applied on the probe, and thus, the robotic arm 116. It will be appreciated that in other embodiments the verification tool 112 may comprise any sensor or tool capable of determining when the robotic arm 116 is contacting the target.

Alternatively, the verification tool 112 may be configured to determine a distance between the verification tool 112 and a target. In such embodiments, the verification tool 112 may comprise a laser sensor configured to measure a distance from the laser sensor to the target. In still other embodiments, the verification tool 112 may comprise ultrasound sensors, optical sensors, or any other sensor or tool capable of measuring a distance to a target.

It will be appreciated that the verification tool 112 may comprise one or more sensors and/or sensors of different modalities.

Data from the verification tool 112 may be provided to a processor of the robot 114, to the processor 104 of the computing device 102, and/or to the navigation system 118. The data may be used to determine when the robotic arm 116 is contacting the target (and thus, transmit instructions to the robotic arm 116 to cause the robotic arm to pause movement). When the robotic arm 116 is no longer moving, data from the robotic arm 116 (such as, for example, pose information obtained from sensors on or integrated with the robotic arm 116) may be used calculate a pose in space of the robotic arm 116 relative to one or more coordinate systems. The calculation may be based not just on data received from the sensor(s), but also on data or information (such as, for example, physical dimensions) about, for example, the robot 114 or a portion thereof, or any other relevant object, which data or information may be stored, for example, in a memory 106 of a computing device 102 or in any other memory. Further, the pose information of the robotic arm 116 may be used to determine a pose of the target using known dimensions of the verification tool 112. For example, a length of the verification tool 112 may be used with the pose information of an end of the robotic arm 116 to determine a pose of the target.

In some embodiments, reference markers (e.g., navigation markers) may be placed on the robot 114 (including, e.g., on the robotic arm 116), or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system and the system can operate without the use of the robot 114 (e.g., with the surgeon manually manipulating one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102 or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300, 400, and/or 500 described herein. The system 100 or similar systems may also be used for other purposes.

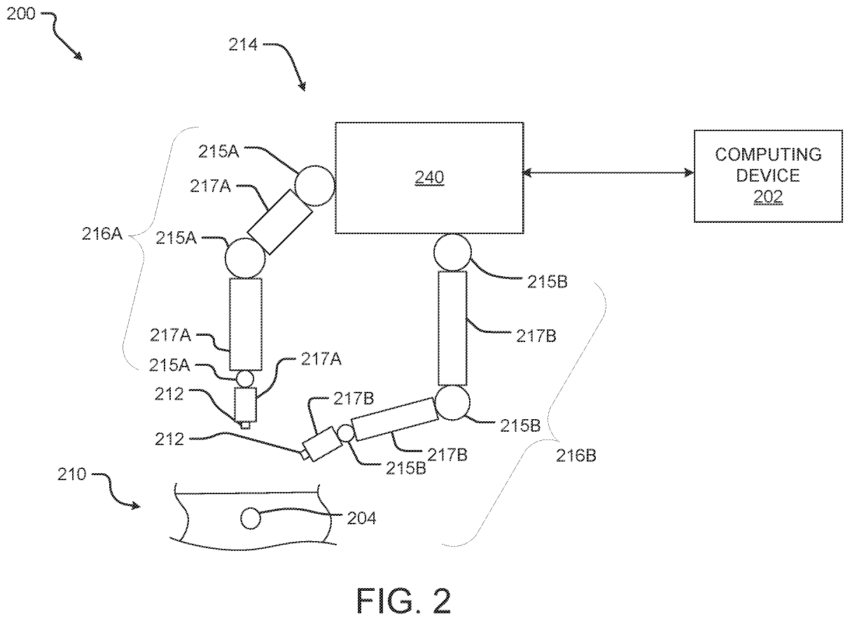

Turning to FIG. 2 a block diagram of a system 200 according to at least one embodiment of the present disclosure is shown. The system 200 includes a computing device 202 (which may be the same as or similar to the computing device 102 described above), and a robot 214 (which may be the same as or similar to the robot 114 described above). The system 200 may be used with the system 100 in some embodiments. Systems according to other embodiments of the present disclosure may comprise more or fewer components than the system 200.

As illustrated, the robot 214 includes one or more robotic arms 216 comprising a first robotic arm 216A (which may comprise one or more members 217A connected by one or more joints 215A) and a second robotic arm 216B (which may comprise one or more members 217B connected by one or more joints 215B), each extending from a base 240. In other embodiments, the robot 214 may include one robotic arm or two or more robotic arms. The base 240 may be stationary or movable. The first robotic arm 216A and/or the second arm 216B is operable to execute one or more planned movements and/or procedures autonomously and/or based on input from a surgeon or user. The first robotic arm 216A and the second robotic arm 216B (and any other additional robotic arms) may operate in a shared or common coordinate space. By operating in the common coordinate space, the first robotic arm 216A and the second robotic arm 216B may avoid colliding with each other during use, as a position of each robotic arm 216A, 216B is known to each other. The first robotic arm 216A and the second robotic arm 216B may also be calibrated relative to each other prior to shipment from a manufacturing facility, during installation, and/or any other time.

In some embodiments, one or more verification tool(s) 212 (which may be the same as or similar to the verification tool 112) may be disposed or supported on an end of the first robotic arm 216A and/or the second robotic arm 216B. In other embodiments, the verification tool 112 may be disposed or secured to any portion of the first robotic arm 216A and/or the second robotic arm 216B. In other embodiments, one or more tools or instruments may be disposed on an end of each of the first robotic arm 216A and the second robotic arm 216B, though the tools or instruments may be disposed on any portion of the first robotic arm 216A and/or the second robotic arm 216B.

In the illustrated example, a target 204 comprises an anatomical element of a patient 210. In other embodiments, the target 204 may comprise the first robotic arm 216A, the second robotic arm 216B, any other robotic arm, an operating table or bed, any component of the system 200, any component external to the system 200, or the like. The verification tool 212 and the first robotic arm 216A, the second robotic arm 216B, or any other robotic arm may be used to determine first pose information, second pose information, and/or any number of pose information of the target 204. The first pose information and the second pose information (and, if applicable, any other number of pose information) of the target 204 may be used to verify a pose of the target 204. In embodiments where the target 204 is the first robotic arm 216A, the second robotic arm 216B, or any other robotic arm, the verified pose may correlate to an accuracy of the calibration of the robotic arm(s) 216A, B. In other embodiments, verification of the pose of the target 204 may verify an accuracy of a registration process. In still other embodiments, the target 204 may be a target anatomical element and verification of the pose of the target anatomical element may confirm that the target anatomical element is in the desired pose. In such embodiments, if the pose of the target anatomical element cannot be verified (e.g., a difference between the first pose information and the second pose information is greater than a pose threshold), then this may indicate that the target anatomical element and/or the operating table may have shifted. In any instance, verification (or lack thereof) of a pose of the target 204 may provide valuable information regarding an accuracy of the system 200 to a surgical team.

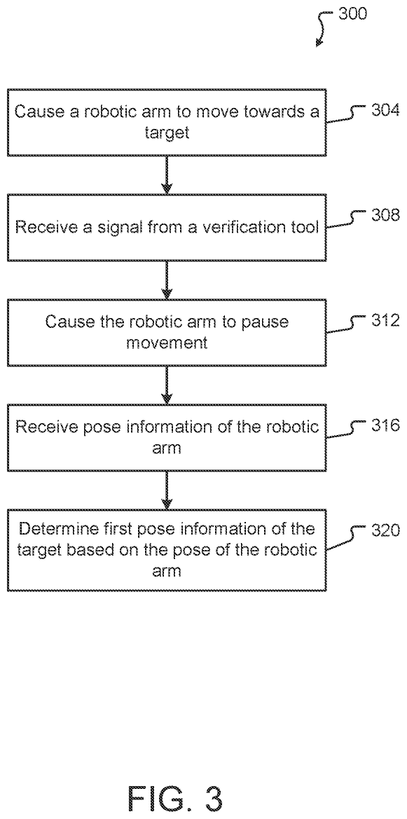

FIG. 3 depicts a method 300 that may be used, for example, for determining first pose information using a verification tool.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in the memory and executed by the processor may cause the processor to execute one or more steps of a function as shown in method 300. One or more portions of a method 300 may be performed by the processor executing any of the contents of memory, such as a registration 120 and/or a verification 122.

The method 300 comprises causing a robotic arm to move towards a target (step 304). The robotic arm may be the same as or similar to the robotic arm 116, 216 of a robot 114, 214 and the target may be the same as or similar to the target 204. The target may comprise another robotic arm, a target anatomical element, an operating table, or any other component of a surgical operating space. In embodiments where the target is another robotic arm, the robotic arm may comprise a first robotic arm such as the first robotic arm 216A and the target may comprise a second robotic arm such as the second robotic arm 216B. The robotic arm may receive instructions to move to a predicted pose of the target, may receive user input (received from, for example, a surgeon or other medical provider) to move towards the target, or may automatically move towards the target along a trajectory. The robotic arm may, in some instances, move towards the target until contact is achieved between the robotic arm and the target as determined by a verification tool such as the verification tool 112, 212.

The verification tool may be disposed on, integrated with, or otherwise coupled to the robotic arm. The verification tool and the robotic arm may together be used to determine pose information of the target. In some embodiments, the verification tool may comprise a probe having a force sensor configured to sense a force applied to the robotic arm. In such embodiments, when the force as detected by the force sensor meets or exceeds a predetermined force threshold, a signal may be generated and transmitted by the verification tool. The force meeting or exceeding the predetermined force threshold may correlate to the robotic arm contacting or touching the target. In other embodiments, the verification tool may comprise other sensors such as, for example, lasers, ultrasound sensors, optical sensors, or the like.

The method 300 also comprises receiving a signal from the verification tool (step 308). A processor such as the processor 104 or any other processor (e.g., a processor of the robot) may receive the signal from the verification tool. The signal may correlate to the robotic arm contacting or touching the target and when such contact or touch is detected, instructions to stop movement of the robotic arm is generated.

The method 300 also comprises causing the robotic arm to pause movement (step 312). Instructions from the processor to stop movement of the robotic arm may be transmitted to the robotic arm. In some embodiments, the instructions may cause one or more motors in the robotic arm to pause or stop movement of the robotic arm. In other embodiments, the instructions may cause one or more brakes to apply a braking force to the one or more motors. In still other embodiments, a combination of causing the motors to stop movement and applying a braking force to the motors may occur.

The method 300 also comprises receiving pose information of the robotic arm (step 316). Data may be received from one or more sensors in the robotic arm by the processor and the data may be used to determine a pose of the robotic arm (or a pose of one or more joints or members of the robotic arm). The one or more sensors may comprise, for example, a position sensor, a proximity sensor, a magnetometer, an accelerometer, a linear encoder, a rotary encoder, or an incremental encoder.

The method 300 also comprises determining first pose information of the target based on the pose of the robotic arm (step 320). Determining the first pose information may also be based on known dimensions of the verification tool. For example, the verification tool may be disposed at an end of the robotic arm and a length of the verification tool may be known. Thus, the length of the verification tool may be added to the pose of the end of the robotic arm to determine the first pose information of the target. In other embodiments, the verification tool may comprise a laser configured to determine a distance between the verification tool and the target and the pose of the verification tool may be based on the pose of the robotic arm. In such embodiments, the distance from the verification tool to the target as determined by the verification tool may be added to the pose of the verification tool to determine the first pose information.

The present disclosure encompasses embodiments of the method 300 that comprise more or fewer steps than those described above, and/or one or more steps that are different than the steps described above. It will also be appreciated that the method 300 or any steps thereof may be repeated as desired.

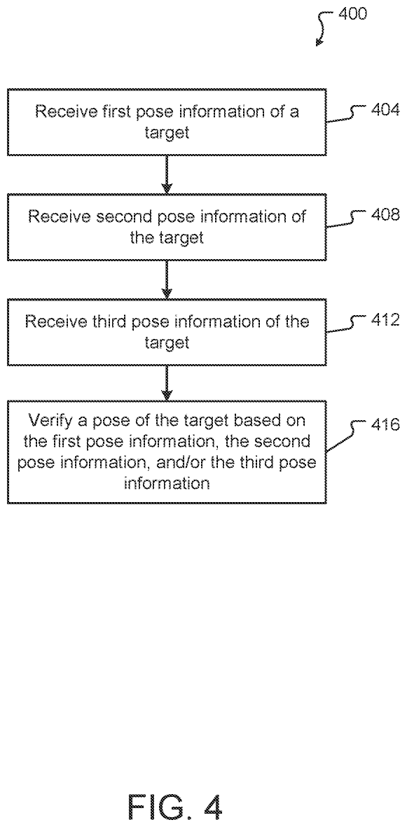

Turning to FIG. 4, a method 400 that may be used, for example, for verifying a pose of a target is depicted.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as a registration 120 and/or a verification 122.

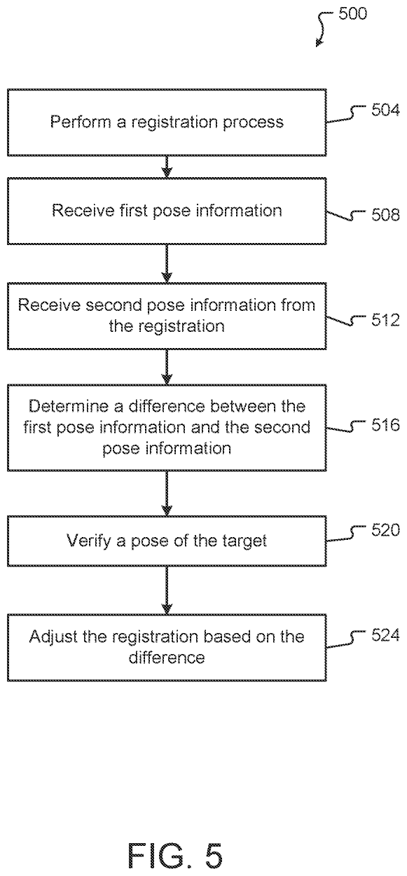

The method 400 comprises receiving first pose information of a target (step 404). The first pose information may be determined using the method 400 as described above, which comprises determining the first pose information using a verification tool such as the verification tool 112, 212 coupled to a robotic arm such as the robotic arm 116, 216. Alternatively, the first pose information may be received from a registration process (such as the registration process as described below in the method 500 of FIG. 5). In still other embodiments, the first pose information may be received from a navigation system such as the navigation system 118.

The method 400 also comprises receiving second pose information of the target (step 408). The step 408 may be the same as or similar to the step 404 described above. The second pose information may be received from the same component or determined by the same process as the first pose information. In other instances, the second pose information may be received from a different component or determined from a different process as the first pose information. For example, the first pose information may be determined from the verification tool and the robotic arm using the method 300 and the second pose information may be received from the registration process of the method 500. In another example—wherein the target may be a second robotic arm—the first pose information may be determined from the verification tool and a first robotic arm using the method 300 and the second pose information may be received from the second robotic arm.

The method 400 also comprises receiving third pose information of the target (step 412). The step 412 may be the same as or similar to the step 404 described above. The third pose information may be received from the same component or determined by the same process as the first pose information and/or the second pose information. In other instances, the third pose information may be received from a different component or determined from a different process as the first pose information and/or the second pose information.

It will be appreciated that in some embodiments the method may not include the step 412.

The method 400 also comprises verifying a pose of the target based on the first pose information, the second pose information, and/or the third pose information (step 416). In some instances, the pose may be verified using the first pose information and the second pose information. In other instances, the pose may be verified using the first pose information, the second pose information, and the third pose information. The processor may execute a verification such as the verification 122 to verify the pose of the target. The verification may enable the processor to verify the pose of the target by determining a difference between the first pose information, the second pose information, and/or the third pose information and determining whether the difference meets a pose threshold. Determining the difference may comprise determining a difference between one or more coordinates and/or an orientation of the first pose information, the second pose information, and/or the third pose information. In other instances, the verification may enable the processor to map and compare the first pose information, the second pose information, and/or the third pose information in space.

In embodiments where the target is a robotic arm, the verified pose may correlate to an accuracy of a calibration of the robotic arm(s). In other embodiments, verification of the pose of the target may verify an accuracy of a registration process. In still other embodiments, the target may comprise a target anatomical element and verification of the pose of the target anatomical element may confirm that the target anatomical element is in the desired pose. In such embodiments, if the pose of the target anatomical element cannot be verified (e.g., a difference between the first pose information and the second pose information is greater than a pose threshold), then this may indicate that the target anatomical element and/or the operating table may have shifted. In any instance, verification (or lack thereof) of a pose of the target may provide valuable information regarding an accuracy of the system to a surgical team.

The present disclosure encompasses embodiments of the method 400 that comprise more or fewer steps than those described above, and/or one or more steps that are different than the steps described above. It will also be appreciated that the method 400 or any steps thereof may be repeated as desired. For example, the method 400 may be repeated prior to a surgical step to determine an accuracy of a pose of a target anatomical element. Similarly, the method 400 may be repeated by a user such as a surgeon or a medical provider to confirm that the patient and/or the operating table have not moved. In other examples, the method 400 may be repeated prior to a start of a surgical operation to verify an accuracy of the robotic calibration.

FIG. 5 depicts a method 500 that may be used, for example, for verifying and/or adjusting a registration.

The method 500 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 500 may be performed by the processor executing any of the contents of memory, such as a registration 120 and/or a verification 122.

The method 500 comprises performing a registration process (step 504). A processor such as the processor 104 or any other processor may execute a registration such as the registration 120 to execute the registration process between a robotic coordinate space and a patient coordinate space. In other embodiments, the registration process may be performed between a navigation coordinate space and a patient coordinate space, between a navigation coordinate space and a robotic coordinate space, and/or between an image coordinate space and one or more of a robotic coordinate space, a patient coordinate space, and/or a navigation coordinate space. During such registration process, the processor may map, transform, or otherwise correlate a particular location with respect to one coordinate space to the particular location with respect to another coordinate space.

The method 500 also comprises receiving first pose information (step 508). The step 508 may be the same as or similar to the step 404 described above.

The method 500 also comprises receiving second pose information from the registration process (step 512). The second pose information may correlate to an expected pose of the target as determined by the registration process.

The method 500 also comprises determining a difference between the first pose information and the second pose information (step 516). Determining the difference may comprise determining a difference between one or more coordinates and/or an orientation of the first pose information and the second pose information. The processor may determine the difference between the first pose information and the second pose information. In some embodiments, the processor may map and compare the first pose information and the second pose information in space.

The method 500 also comprises verifying a pose of the target (step 520). The pose of the target may be verified when the difference as determined in, for example, the step 516 is less than a pose threshold. The pose threshold may correlate to a maximum allowable difference between the first pose information and the second pose information (or any other number of pose information), or any portion of the first pose information and the second pose information. The pose of the target may be verified when the difference is less than the pose threshold.

The pose threshold may be determined automatically using artificial intelligence and training data (e.g., historical cases) in some embodiments. In other embodiments, the pose threshold may be or comprise, or be based on, surgeon input received via the user interface. In further embodiments, the pose threshold may be determined automatically using artificial intelligence, and may thereafter be reviewed and approved (or modified) by a surgeon or other user. In some embodiments, a notification may be generated when the difference meets or exceeds the pose threshold. In examples where the difference comprises a plurality of differences (e.g., a difference in one or more coordinates and/or a difference in the orientation of the first pose information and the second pose information), a notification may be generated for each difference that meets or exceeds the corresponding threshold difference. The notification may alert a surgeon or user of an expected difference that the surgeon or other user may wish to avoid or otherwise mitigate.

It will be appreciated that in some embodiments if the step 520 does not occur (because, for example, the pose of the target cannot be verified due to the difference meeting or exceeding the pose threshold), then the step 524 (described below) may occur.

The method 500 also comprises adjusting the registration based on the difference (step 524). In embodiments where pose of the target cannot be verified in the step 520, the registration may be adjusted by adjusting the pose of the target based on the difference. For example, in embodiments where the target comprises a target anatomical element, a registration pose of the target anatomical element may be shifted based on the difference. Re-registration, which is time intensive, may be avoided. Further, by reducing the number of registrations processes and/or eliminating re-registration, radiation exposure to a patient is also reduced. Thus, the method 500 (and any of the methods 300 and/or 400) increases safety of the patient and the surgical team by confirming an accuracy of a robot calibration, a registration process, and a pose of a target anatomical element.

The present disclosure encompasses embodiments of the method 500 that comprise more or fewer steps than those described above, and/or one or more steps that are different than the steps described above. It will also be appreciated that the method 500 or any steps thereof may be repeated as desired.

As noted above, the present disclosure encompasses methods with fewer than all of the steps identified in FIGS. 3, 4, and 5 (and the corresponding description of the methods 300, 400, and 500), as well as methods that include additional steps beyond those identified in FIGS. 3, 4, and 5 (and the corresponding description of the methods 300, 400, and 500). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for verifying a pose of a target during a surgical operation comprising:
a robotic arm;
a verification tool coupled to the robotic arm;
a processor; and
a memory storing data for processing by the processor, the data, when processed, causes the processor to:
cause the robotic arm to contact a target with the verification tool to yield first pose information of the target;
receive second pose information of the target; and
verify the pose of the target based on the first pose information and the second pose information.

2. The system of claim 1, wherein the second pose information of the target is received from another robotic arm or a registration process.

3. The system of claim 2, wherein the memory stores further data for processing by the processor that, when processed, causes the processor to:
perform the registration process;
receive the second pose information from the registration process;
determine a difference between the first pose information and the second pose information of the target; and
adjust the registration by shifting the second pose information of the target based on the difference.

4. The system of claim 2, wherein the memory stores further data for processing by the processor that, when processed, causes the processor to:
perform the registration process;
receive the second pose information from the registration process; and
verify the registration based on the first pose information and the second pose information.

5. The system of claim 1, wherein the target comprises another robotic arm or a target anatomical element.

6. The system of claim 1, wherein verifying the pose of the target verifies a calibration of the robotic arm.

7. The system of claim 1, wherein the verification tool comprises a probe.

8. The system of claim 7, wherein the probe is configured to sense when the robotic arm contacts the target, and wherein the memory stores further data for processing by the processor that, when processed, causes the processor to:
cause the robotic arm to move towards the target;
receive a signal from the probe corresponding to the probe contacting the target;
cause the robotic arm to pause movement;
receive pose information of the robotic arm; and
determine the first pose information of the target based on the pose information of the robotic arm.

9. The system of claim 8, wherein the probe comprises a force sensor and wherein the probe sends a signal corresponding to the probe contacting the target when the force sensor measures a force that meets a predetermined force threshold.

10. The system of claim 8, wherein determining the first pose information comprises combining the pose of the robotic arm with a known length of the probe.

11. The system of claim 1, wherein the memory stores further data for processing by the processor that, when processed, causes the processor to
receive third pose information; and
verify the pose of the target based on the first pose information, the second pose information, and the third pose information.

12. The system of claim 1, wherein verifying the pose of the target occurs prior to a surgical step.

13. A system for verifying a pose of a target during a surgical operation comprising:
a verification tool;
a processor; and
a memory storing data for processing by the processor, the data, when processed, causes the processor to:
receive first pose information of the target using the verification tool;
receive second pose information of the target; and
verifying the pose of the target based on the first pose information and the second pose information.

14. The system of claim 13, wherein the target comprises a robotic arm or a target anatomical element.

15. The system of claim 13, wherein the verification tool comprises a probe.

16. The system of claim 15, wherein determining the first pose information comprises combining the pose of the robotic arm with a known length of the probe.

17. The system of claim 13, wherein the memory stores further data for processing by the processor that, when processed, causes the processor to
receive third pose information; and
verify the pose of the target based on the first pose information, the second pose information, and the third pose information.

18. The system of claim 13, wherein the memory stores further data for processing by the processor that, when processed, causes the processor to:
perform a registration process;
receive the second pose information from the registration process;
determine a difference between the first pose information and the second pose information of the target; and
adjust the registration by shifting the second pose information of the target based on the difference.

19. The system of claim 13, wherein the memory stores further data for processing by the processor that, when processed, causes the processor to:
perform a registration process;
receive the second pose information from the registration process; and
verify the registration based on the first pose information and the second pose information.

20. A system for verifying a pose of a target comprising:
a processor; and
a memory storing data for processing by the processor, the data, when processed, causes the processor to:
cause a robotic arm to contact a target with a verification tool to yield first pose information of the target;
cause the robotic arm to contact a target with the verification tool to yield second pose information of the target;
cause the robotic arm to contact a target with the verification tool to yield third pose information of the target; and
verifying the pose of the target based on the first pose information, the second pose information, and the third pose information.
